(19) United States
(12) Patent Application Publication
(10) Pub. No.: US 2017/0232193 A1
(43) Pub. Date: Aug. 17, 2017

(54) Diagnostic Transceiver Assembly

(71) Applicant: Paul Gargano, West Hyannisport, MA (US)

(72) Inventor: Paul Gargano, West Hyannisport, MA (US)

(21) Appl. No.: 15/041,463

(22) Filed: Feb. 11, 2016

(51) Int. Cl. A61M 5/172; A61B 5/145; A61B 5/0205; A61M 5/142; A61B 5/024

(52) U.S. Cl. CPC A61M 5/1723; A61M 5/14276; A61B 5/024; A61B 5/0205; A61B 5/14532; A61M 2202/0007; A61M 2230/20; A61M 2205/04; A61M 2205/3523; A61M 2205/52; A61M 2205/50; A61M 2205/8206

(57) ABSTRACT

A diagnostic transceiver assembly includes a diagnostic unit that may be implanted into a human body. The diagnostic unit monitors physiological functions of the human body to include heart rate and blood serum levels. The diagnostic unit generates a diagnostic sequence when the diagnostic unit detects blood serum levels outside of a trigger ratio. A dispensing unit may be implanted into the human body. The dispensing unit is in fluid communication with the human circulatory system. The dispensing unit contains insulin. The dispensing unit releases a measured amount of the insulin into the human circulatory system in response to the diagnostic sequence.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING ASSEMBLY.

Not Applicable

### STATEMENT REGARDING PRIOR DISCLOSURES BY THE INVENTOR OR JOINT INVENTOR

Not Applicable

### BACKGROUND OF THE INVENTION

(1) Field of the Invention

(2) Description of Related Art including Information Disclosed under 37 CFR 1.97 and 1.98.

The disclosure and prior art relates to diagnostic devices and more particularly pertains to a new diagnostic device for monitoring and treating Type 1 Diabetes and Type 2 Diabetes.

### BRIEF SUMMARY OF THE INVENTION

An embodiment of the disclosure meets the needs presented above by generally comprising a diagnostic unit that may be implanted into a human body. The diagnostic unit monitors physiological functions of the human body to include heart rate and blood serum levels. The diagnostic unit generates a diagnostic sequence when the diagnostic unit detects blood serum levels outside of a trigger ratio. A dispensing unit may be implanted into the human body. The dispensing unit is in fluid communication with the human circulatory system. The dispensing unit contains insulin. The dispensing unit releases a measured amount of the insulin into the human circulatory system in response to the diagnostic sequence.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a diagnostic transceiver assembly according to an embodiment of the disclosure.

FIG. 2 is a perspective in-use view of an embodiment of the disclosure.

FIG. 3 is a schematic view of an embodiment of the disclosure.

### DETAILED DESCRIPTION OF THE INVENTION

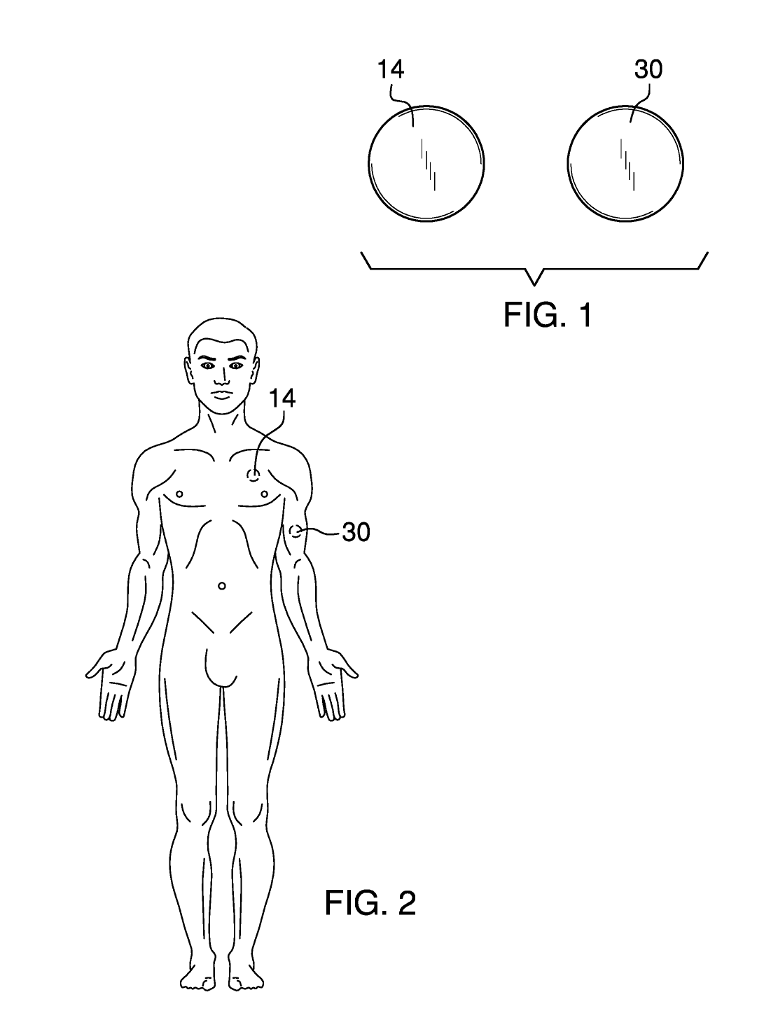

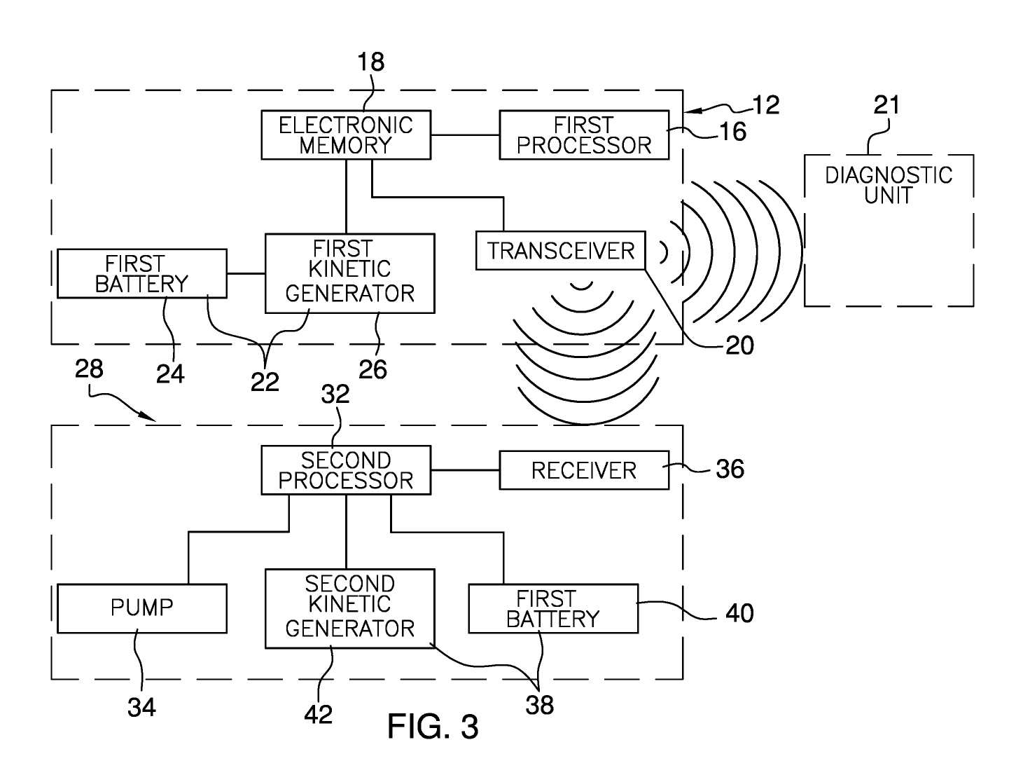

With reference now to the drawings, and in particular to FIGS. 1 through 3 thereof, a new diagnostic device embodying the principles and concepts of an embodiment of the disclosure and generally designated by the reference numeral 10 will be described.

As best illustrated in FIGS. 1 through 3, the diagnostic transceiver assembly 10 generally comprises a diagnostic unit 12 that may be implanted into a human body. The diagnostic unit 12 monitors physiological functions of the human body to include heart rate and blood serum levels. The diagnostic unit 12 generates a diagnostic sequence when the diagnostic unit 12 detects blood serum levels outside of a trigger ratio. Gargano et al., U.S. Pat. No. 5,629,678, discloses a means and method for locating, tracking and recovering humans in distress. The diagnostic unit 12 will function according to U.S. Pat. No. 5,629,678 and will incorporate improvements described henceforth.

The diagnostic unit 12 comprises a first housing 14 that may be implanted into the human body. The first housing 14 is comprised of a biologically inert material or the like such as titanium. A first processor 16 is positioned within the first housing 14 and the first processor 16 selectively generates the diagnostic sequence. The first processor 16 may comprise an electronic processor or the like. The first processor 16 includes an electronic memory 18. The electronic memory 18 stores a database containing data for all known maladies and illnesses related to the monitored physiological functions.

A transceiver 20 is positioned within the first housing 14 and the transceiver 20 is electrically coupled to the first processor 16. The transceiver 20 is in electromagnetic communication and fluid communication with the human body. Thus, the transceiver 20 may receive a diagnostic signal relating to a human heart beat. Additionally, the transceiver 20 may receive a diagnostic signal relating to blood serum chemistry. Specifically monitored aspects of the blood serum chemistry may include, but not be limited to, red blood cell count, white blood cell count and blood sugar levels. The transceiver 20 may include a continuous glucose monitor (CGM) or the like.

The transceiver 20 communicates the diagnostic signal to the first processor 16. The diagnostic signal contains data pertaining to the monitored physiological functions. The diagnostic signal may include data specifically related to red blood cell count, white blood cell count and blood sugar levels. Thus, the data may correspond to illnesses related to red and white blood cell count, Type 1 Diabetes and Type 2 Diabetes. The data contained in the diagnostic signal is compared against the data in the electronic memory 18. The first processor 16 generates the diagnostic sequence when the diagnostic signal indicates the monitored blood serum chemistry does not correspond with the data in the electronic memory 18.

The transceiver 20 may be in electrical communication with an extrinsic electronic device 21. The extrinsic electronic device 21 may comprise a diagnostic tool in a medical facility. Additionally, the extrinsic electronic device 21 may comprise a display, an audio device, or other electronic multimedia device. The transceiver 20 may communicate the diagnostic signal to the extrinsic electronic device 21. Thus, the extrinsic electronic device 21 may communicate the diagnostic signal to an observer via an electronic image, audio or other means of multimedia communication. The extrinsic electronic device may

A first power supply 22 is positioned within the first housing 14 and the first power supply 22 is electrically coupled to the first processor 16. The first power supply 22 comprises at least one first battery 24 and a first kinetic generator 26. The first kinetic generator 26 is in kinetic communication with the human body. Thus, the first kinetic generator 26 may produce an electrical current derived from motion of the human body. The first kinetic generator 26 is electrically coupled to the at least one first battery 24. Thus, the first kinetic generator 26 continuously charges the at least one first battery 24.

A dispensing unit 28 is provided and the dispensing unit 28 may be implanted into the human body thereby. Thus, the dispensing unit 28 is in fluid communication with the human circulatory system. The dispensing unit 28 is in electrical communication with the diagnostic unit 12. The dispensing unit 28 contains insulin. The dispensing unit 28 releases a measured amount of the insulin into the human circulatory system in response to the diagnostic sequence.

The dispensing unit 28 comprises a second housing 30 that may be implanted within the human body. The second housing 30 is comprised of a biologically inert material or the like such as titanium. A second processor 32 is positioned within the second housing 30. The second processor 32 may comprise an electronic processor or the like. A pump 34 is positioned within the second housing 30 and the pump 34 is electrically coupled to the second processor 32.

The pump 34 contains the insulin. The pump 34 is in fluid communication with the human circulatory system through any conventional means. Thus, the pump 34 may deliver the measured amount of insulin into the human circulatory system. The pump 34 may comprise a miniature insulin pump or the like.

A receiver 36 is positioned within the second housing 30 and the receiver 36 is electrically coupled to the second processor 32. The receiver 36 is in electrical communication with the transceiver 20. Thus, the second processor 32 receives the diagnostic sequence from the first processor 16. The receiver 36 may comprise a radio frequency receiver or the like. The second processor 32 actuates the pump 34 to deliver the measured amount of the insulin when the first processor 16 generates the diagnostic sequence. The diagnostic sequence may include prescribed insulin treatments corresponding to Type 1 Diabetes and Type 2 Diabetes.

A second power supply 38 is positioned within the second housing 30 and the second power supply 38 is electrically coupled to the second processor 32. The second power supply 38 comprises at least one second battery 40 and a second kinetic generator 42. The second kinetic generator 42 is in kinetic communication with the human body. Thus, the second kinetic generator 42 produces an electrical current derived from motion of the human body. The second kinetic generator 42 is electrically coupled to the at least one second battery 40. Thus, the second kinetic generator 42 continuously charges the at least one second battery 40.

In use, each of the first housing 14 and the second housing 30 are implanted into the human body. The transceiver 20 continuously communicates the diagnostic signal to the first processor 16. The first processor 16 generates the diagnostic sequence when the diagnostic signal includes data pertaining to improper blood sugar levels. The pump 34 releases a prescribed amount of the insulin in response to the diagnostic sequence. Thus, the symptoms of Type 1 Diabetes and Type 2 Diabetes are treated. The transceiver 20 communicates the diagnostic signal to the extrinsic electronic device 21. Thus, a medical professional may offer a diagnosis and corrective action related to the diagnosis.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A diagnostic transceiver assembly being configured to be implanted into a human body, said assembly comprising:
a diagnostic unit being configured to be implanted into a human body thereby facilitating said diagnostic unit to monitor physiological functions of the human body to include heart rate and blood serum levels, said diagnostic unit generating a diagnostic sequence when said diagnostic unit detects blood serum levels outside of a trigger ratio; and
a dispensing unit being configured to be implanted into the human body thereby facilitating said dispensing unit to be in fluid communication with the human circulatory system, said dispensing unit being in electrical communication with said diagnostic unit, said dispensing unit being configured to contain insulin thereby facilitating said dispensing unit to release a measured amount of the insulin into the human circulatory system in response to said diagnostic sequence.

2. The assembly according to claim 1, wherein said diagnostic unit comprises:
a first housing being configured to be implanted into the human body; and
a first processor being positioned within said first housing, said first processor selectively generating said diagnostic sequence, said first processor including an electronic memory.

3. The assembly according to claim 2, further comprising a transceiver being positioned within said first housing, said transceiver being electrically coupled to said first processor, said transceiver being configured to be in electromagnetic communication with the human body thereby facilitating said transceiver to receive an diagnostic signal relating to a human heart beat and to receive a diagnostic signal relating to blood serum chemistry.

4. The assembly according to claim 3, wherein said transceiver communicates the diagnostic signal to said first processor, said first processor generating said diagnostic sequence when the diagnostic signal indicates the blood serum chemistry is not within the trigger ratio.

5. The assembly according to claim 2, further comprising a first power supply being positioned within said first housing, said first power supply being electrically coupled to said first processor.

6. The assembly according to claim 5, wherein said first power supply comprises:
at least one first battery; and
a first kinetic generator being configured to be in kinetic communication with the human body thereby facilitating said first kinetic generator to produce an electrical current derived from motion of the human body, said first kinetic generator being electrically coupled to said at least one first battery such that said first kinetic generator continuously charges said at least one first battery.

7. The assembly according to claim 1, wherein said dispensing unit comprises:
a second housing being configured to be implanted within the human body; and
a second processor being positioned within said second housing.

8. The assembly according to claim 7, further comprising a pump being positioned within said second housing, said pump being electrically coupled to said second processor, said pump being configured to contain the insulin, said pump being configured to be in fluid communication with the human circulatory system thereby facilitating said pump to deliver the measured amount into the human circulatory system.

9. The assembly according to claim 8, further comprising:
a transceiver;
a first processor generating a diagnostic sequence; and
a receiver being positioned within said second housing, said receiver being electrically coupled to said second processor, said receiver being in electrical communication with said transceiver such that said second processor receives said diagnostic sequence from said first processor, said second processor actuating said pump to deliver the measured amount of the insulin when said first processor generates said diagnostic sequence.

10. The assembly according to claim 7, wherein a second power supply being positioned within said second housing, said second power supply being electrically coupled to said second processor.

11. The assembly according to claim 10, wherein said second power supply comprises:
at least one second battery; and
a second kinetic generator being configured to be in kinetic communication with the human body thereby facilitating said second kinetic generator to produce an electrical current derived from motion of the human body, said second kinetic generator being electrically coupled to said at least one second battery such that said second kinetic generator continuously charges said at least one second battery.

12. A diagnostic transceiver assembly being configured to be implanted into a human body, said assembly comprising:
a diagnostic unit being configured to be implanted into a human body thereby facilitating said diagnostic unit to monitor physiological functions of the human body to include heart rate and blood serum levels, said diagnostic unit generating a diagnostic sequence when said diagnostic unit detects blood serum levels outside of a trigger ratio, said diagnostic unit comprising:
a first housing being configured to be implanted into the human body,
a first processor being positioned within said first housing, said first processor selectively generating said diagnostic sequence, said first processor including an electronic memory,
a transceiver being positioned within said first housing, said transceiver being electrically coupled to said first processor, said transceiver being configured to be in electromagnetic communication and fluid communication with the human body thereby facilitating said transceiver to receive an diagnostic signal relating to a human heart beat and to receive a diagnostic signal relating to blood serum chemistry, said transceiver communicating the diagnostic signal to said first processor, said first processor generating said diagnostic sequence when the diagnostic signal indicates the blood serum chemistry is not within the trigger ratio,
a first power supply being positioned within said first housing, said first power supply being electrically coupled to said first processor, said
first power supply comprising:
at least one first battery, and
a first kinetic generator being configured to be in kinetic communication with the human body thereby facilitating said first kinetic generator to produce an electrical current derived from motion of the human body, said first kinetic generator being electrically coupled to said at least one first battery such that said first kinetic generator continuously charges said at least one first battery; and
a dispensing unit being configured to be implanted into the human body thereby facilitating said dispensing unit to be in fluid communication with the human circulatory system, said dispensing unit being in electrical communication with said diagnostic unit, said dispensing unit being configured to contain insulin thereby facilitating said dispensing unit to release a measured amount of the insulin into the human circulatory system in response to said diagnostic sequence, said dispensing unit comprising:
a second housing being configured to be implanted within the human body,
a second processor being positioned within said second housing,
a pump being positioned within said second housing, said pump being electrically coupled to said second processor, said pump being configured to contain the insulin, said pump being configured to be in fluid communication with the human circulatory system thereby facilitating said pump to deliver the measured amount into the human circulatory system,
a receiver being positioned within said second housing, said receiver being electrically coupled to said second processor, said receiver being in electrical communication with said transceiver such that said second processor receives said diagnostic sequence from said first processor, said second processor actuating said pump to deliver the measured amount of the insulin when said first processor generates said diagnostic sequence, and
a second power supply being positioned within said second housing, said second power supply being electrically coupled to said second processor, said second power supply comprising:
at least one second battery, and
a second kinetic generator being configured to be in kinetic communication with the human body thereby facilitating said second kinetic generator to produce an electrical current derived from motion of the human body, said second kinetic generator being electrically coupled to said at least one second battery such that said second kinetic generator continuously charges said at least one second battery.
